(19) United States
(12) Patent Application Publication
(10) Pub. No.: US 2020/0375576 A1
(43) Pub. Date: Dec. 3, 2020

(54) CO-REGISTRATION SYSTEMS AND METHODS FO RENHANCING THE QUALITY OF INTRAVASCULAR IMAGES

(71) Applicant: PHILIPS IMAGE GUIDED THERAPY CORPORATION, San Diego, CA (US)

(72) Inventor: Kellen MOULTON, SAN MARCOS, CA (US)

(21) Appl. No.: 15/931,634

(22) Filed: May 14, 2020

Related U.S. Application Data
(60) Provisional application No. 62/856,040, filed on Jun. 1, 2019.

(51) Int. Cl. A61B 8/00; A61B 6/00; A61B 8/12; A61B 8/08; G06T 7/00

(52) U.S. Cl. CPC A61B 8/54; A61B 6/504; A61B 6/5247; A61B 8/12; G06T 2207/30004; A61B 8/4254; A61B 8/463; G06T 7/0012; G06T 2207/10132; A61B 8/5261

(57) ABSTRACT

In an exemplary embodiment, a co-registration system according to the embodiments disclosed herein includes one or more processors, an intravascular ultrasound (IVUS) device including an IVUS imaging probe, an angiogram device, and memory storing instructions. The IVUS device and angiogram device are in communication with the one or more processors. The instructions, when executed by the one or more processors, cause the one or more processors to determine a pull-back speed of the IVUS imaging probe based on received radiological image data from the angiogram device. In addition, the one or more processors determine a beamforming setting for the INTS imaging probe based on the pull-back speed, wherein the determined beamforming setting changes an image quality parameter for a subsequently received IVUS image. And, the one or more processors provide the determined beamforming setting to the IVUS imaging probe.

### CROSS-REFERENCE TO RELATED APPLICATION

This application claims priority to U.S. Provisional Application Ser. No. 62/856,040, filed Jun. 1, 2019.

### FIELD OF THE DISCLOSURE

The systems and devices described herein generally relate to imaging blood vessels. More particularly, the present invention is directed to methods and systems for generating composite displays relating a first image rendered from a first type of data and a second image rendered from a second type of data. A particular example of such composite display comprises an angiogram displayed along-side an IVUS image.

### BACKGROUND

In coronary arteries, vascular diseases including vessel lumen narrowing, usually due to atherosclerotic plaque, can lead to reduced blood flow to a heart muscle, angina (chest pain) and myocardial infarction—a heart attack. A variety of interventional treatments of cardiovascular disease are presently available to identify and treat such narrowing of a vessel lumen. Examples of such treatments include balloon angioplasty and/or deployment of stents. Diagnostic imaging is utilized to identify the extent and/or type of blockages within vessels prior to and/or during the treatment of such blockages. Diagnostic imaging also enables doctors to ensure proper treatment of diseased vessels and verify the efficacy of such treatment.

Co-registration systems typically include at least an intravascular ultrasound (IVUS) device to collect ultrasound image data and an angiogram/fluoroscopy device to collect radiological image data. After collecting the data, the co-registration systems generate diagnostic images to diagnose and/or treat problems within the blood vessels. For example, the IVUS device uses a series of ultrasound pulses to create cross-sectional images of the vessels. However, it may difficult to discern the location where the IVUS image is taken using the IVUS device alone. As such, the co-registration system uses the angiogram device to map the IVUS image or a pull-back recording (e.g., multiple image frames and/or video) to a location on an angiogram image with high precision. In other words, by observing the position of the radiopaque markers on an imaging probe for the system, the system can sync the IVUS and angiograms images to both detect problems within the vessels and the location where the image was taken.

In the typical operation of the IVES device, after pivoting the imaging probe to a region of interest, the user pulls back the imaging probe. During the pull-back, the imaging probe emits the ultrasound pulses and then receives the echoes of these emitted pulses. The co-registration system takes these echoes to generate the cross-sectional image of the vessel. However, in some instances, the user and/or system may pull back probe faster or slower depending on the circumstance, which may cause the image quality to vary, including deteriorate. Deterioration of the image quality may cause the user increased difficulty in diagnosing and/or treating vascular problems/diseases. Accordingly, it is desirable to provide one or more systems and/or methods to address these and other shortcomings.

### SUMMARY

Exemplary embodiments disclosed herein include, but are not limited to, the following examples.

In at least one exemplary embodiment, a medical imaging system including a co-registration system includes one or more processors, an intravascular ultrasound (IVUS) device including an IVUS imaging probe, an angiogram device, and memory storing instructions. The IVUS device and angiogram device are in communication with the one or more processors. The instructions, when executed by the one or more processors, cause the processors to determine a pull-back speed of the IVUS imaging probe based on received radiological image data from the angiogram device, determine a beamforming setting for the IVUS imaging probe based on the pull-back speed, and provide the determined beamforming setting to the IVUS imaging probe. The determined beamforming setting changes an image quality parameter for a subsequently received IVUS image.

In another exemplary embodiment, the medical imaging system including the co-registration system according to the previous paragraph, wherein the IVUS imaging probe comprises one or more radiopaque markers, wherein the received radiological image data comprises first radiological image data indicating a first location of the one or more radiopaque markers and second radiological image data indicating a second location of the one or more radiopaque markers, and wherein the determining the pull-back speed is based on the first location and the second location.

In another exemplary embodiment, the medical imaging system including the co-registration system according to the previous paragraph, wherein the determining the pull-back speed of the IVUS imaging probe comprises determining the pull-back speed based on determining a distance between the first location and the second location.

In another exemplary embodiment, the medical imaging system including the co-registration system according to the previous paragraph, wherein the memory storing instructions that when executed by the one or more processors, further cause the one or more processors to receive a user input indicating the pull-back speed and wherein the determining the pull-back speed is based on the user input.

In another exemplary embodiment, the medical imaging system including the co-registration system according to any of the previous paragraphs, wherein the beamforming setting indicates a number and sequence of ultrasound pulses emitted by the IVUS imaging probe and wherein the image quality parameter for the subsequently received IVUS image is based the number and sequence of the ultrasound pulses emitted by the IVUS imaging probe.

In another exemplary embodiment, the medical imaging system including the co-registration system according to any of the previous paragraphs, wherein the image quality parameter comprises at least one of frame rate, resolution, and depth of penetration of a vessel.

In another exemplary embodiment, the medical imaging system including the co-registration system according to any of the previous paragraphs, wherein the determining the beamforming setting comprises determining a first beamforming setting based on a first pullback speed, wherein the first beamforming setting indicates a first number of ultrasound pulses emitted by the IVUS imaging probe.

In another exemplary embodiment, the medical imaging system including the co-registration system according to any of the previous paragraphs, wherein the determining the beamforming setting comprises determining a second beamforming setting based on a second pullback speed, wherein the second pullback speed is slower than the first pullback speed, wherein the second beamforming setting indicates a second number of ultrasound pulses emitted by the IVUS imaging probe, and wherein the second number of ultrasound pulses is greater than the first number of ultrasound pulses.

In another exemplary embodiment, the medical imaging system including the co-registration system according to any of the previous paragraphs, further comprising a display device, and wherein the memory storing instructions that when executed by the one or more processors, further cause the one or more processors to receive subsequently received IVUS image data based on the determined beamforming setting, generate the subsequently received IVUS image from the subsequently received IVUS image, generate a subsequently received radiological image corresponding to the subsequently received IVUS image, and provide for display on the display device, the subsequently received IVUS image and the subsequently received radiological image.

In another exemplary embodiment, a non-transitory computer readable medium storing instructions for execution by a processor incorporated into a medical imaging system including a co-registration system, wherein execution of the instructions by the processor cause the processor to determine a pull-back speed of an intravascular ultrasound (IVUS) imaging probe based on received radiological image data from an angiogram device; determine a beamforming setting for the IVUS imaging probe based on the pull-back speed, wherein the determined beamforming setting changes an image quality parameter for a subsequently received IVUS image; and provide the determined beamforming setting to the IVUS imaging probe.

In another exemplary embodiment, the non-transitory computer readable medium of the preceding paragraph, wherein the POLS imaging probe comprises one or more radiopaque markers, wherein the received radiological image data comprises first radiological image data indicating a first location of the one or more radiopaque markers and second radiological image data indicating a second location of the one or more radiopaque markers, and wherein the determining the pull-back speed is based on the first location and the second location.

In another exemplary embodiment, the non-transitory computer readable medium of the preceding paragraph, wherein when determining the pull-back speed of the IVUS imaging probe, the instructions cause the processor to: determine the pull-back speed based on determining a distance between the first location and the second location.

In another exemplary embodiment, the non-transitory computer readable medium of any of the preceding paragraphs, the instructions further causing the processor to: receive a user input indicating the pull-back speed, and wherein the pull-back speed is based on the user input.

In another exemplary embodiment, the non-transitory computer readable medium of any of the preceding paragraphs, wherein the beamforming setting indicates a number and sequence of ultrasound pulses emitted by the IVUS imaging probe, and wherein the image quality parameter for the subsequently received IVUS image is based the number and sequence of the ultrasound pulses emitted by the IVUS imaging probe.

In another exemplary embodiment, the non-transitory computer readable medium of any of the preceding paragraphs, wherein the image quality parameter comprises at least one of frame rate, resolution, and depth of penetration of a vessel.

In another exemplary embodiment, the non-transitory computer readable medium of any of the preceding paragraphs, wherein when determining the beamforming setting, the instructions cause the processor to determine a first beamforming setting based on a first pullback speed, wherein the first beamforming setting indicates a first number of ultrasound pulses emitted by the IVUS imaging probe.

In another exemplary embodiment, the non-transitory computer readable medium of the preceding paragraph, wherein when determining the beamforming setting, the instructions cause the processor to determine a second beamforming setting based on a second pullback speed, wherein the second pullback speed is slower than the first pullback speed, wherein the second beamforming setting indicates a second number of ultrasound pulses emitted by the IVUS imaging probe, and wherein the second number of ultrasound pulses is greater than the first number of ultrasound pulses.

In another exemplary embodiment, the non-transitory computer readable medium of the preceding paragraph, wherein when determining the beamforming setting, the instructions cause the processor to determine a third beamforming setting based on a pullback speed indicating substantially zero, wherein the third beamforming setting indicates an optimal number of ultrasound pulses emitted by the IVUS imaging probe, and wherein the optimal number of ultrasound pulses is greater than the first number of ultrasound pulses and the second number of ultrasound pulses.

In another exemplary embodiment, the non-transitory computer readable medium of any of the preceding paragraphs, the instructions further causing the processor to: receive subsequently received IVUS image data based on the determined beamforming setting; generate the subsequently received IVUS image from the subsequently received IVUS image; generate a subsequently received radiological image corresponding to the subsequently received IVUS image; and provide for display on a display device, the subsequently received IVUS image and the subsequently received radiological image.

In another exemplary embodiment, a method to be performed by a processor incorporated into a medical imaging system including a co-registration system comprises: determining a pull-back speed of an intravascular ultrasound (IVUS) imaging probe based on received radiological image data from an angiogram device; determining a beamforming setting for the IVUS imaging probe based on the pull-back speed, wherein the determined beamforming setting changes an image quality parameter for a subsequently received IVUS image; and providing the determined beamforming setting to the IVUS imaging probe.

In another exemplary embodiment, the method according to the previous paragraph, wherein the IVUS imaging probe comprises one or more radiopaque markers, wherein the received radiological image data comprises first radiological image data indicating a first location of the one or more radiopaque markers and second radiological image data indicating a second location of the one or more radiopaque markers, and wherein the determining the pull-back speed is based on the first location and the second location.

In another exemplary embodiment, the method according to any of the previous paragraphs wherein the determining the pull-back speed of the IVUS imaging probe comprises: determining the pull-back speed based on determining a distance between the first location and the second location.

In another exemplary embodiment, the method according to any of the previous paragraphs further comprising: receiving a user input indicating the pull-back speed, and wherein the determining the pull-back speed is based on the user input.

In another exemplary embodiment, the method according to any of the previous paragraphs, wherein the beamforming setting indicates a number and sequence of ultrasound pulses emitted by the IVUS imaging probe, and wherein the image quality parameter for the subsequently received IVUS image is based the number and sequence of the ultrasound pulses emitted by the IVUS imaging probe.

In another exemplary embodiment, the method according to any of the previous paragraphs, wherein the image quality parameter comprises at least one of frame rate, resolution, and depth of penetration of a vessel.

In another exemplary embodiment, the method according to any of the previous paragraphs wherein the determining the beamforming setting comprises determining a first beamforming setting based on a first pullback speed, wherein the first beamforming setting indicates a first number of ultrasound pulses emitted by the IVUS imaging probe.

In another exemplary embodiment, the method according to any of the previous paragraphs wherein the determining the beamforming setting comprises determining a second beamforming setting based on a second pullback speed, wherein the second pullback speed is slower than the first pullback speed, wherein the second beamforming setting indicates a second number of ultrasound pulses emitted by the IVUS imaging probe, and wherein the second number of ultrasound pulses is greater than the first number of ultrasound pulses.

In another exemplary embodiment, the method according to any of the previous paragraphs wherein the determining the beamforming setting comprises determining a third beamforming setting based on a pullback speed indicating substantially zero, wherein the third beamforming setting indicates an optimal number of ultrasound pulses emitted by the IVUS imaging probe, and wherein the optimal number of ultrasound pulses is greater than the first number of ultrasound pulses and the second number of ultrasound pulses.

In another exemplary embodiment, the method according to any of the previous paragraphs, further comprising: receiving subsequently received IVUS image data based on the determined beamforming setting; generating the subsequently received IVUS image from the subsequently received IVUS image; generating a subsequently received radiological image corresponding to the subsequently received IVUS image; and providing for display on a display device, the subsequently received IVUS image and the subsequently received radiological image.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to he construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic illustration of a system for implementing catheter image co-registration;

FIG. 2 depicts an illustrative angiogram image;

FIG, 3 depicts an illustrative fluoroscopic image of a radiopaque marker mounted upon a catheter;

FIG. 4 depicts an illustrative radiological image along-side a cross-sectional IVUS image;

FIG. 5 depicts an illustrative radiological image along-side a cross-sectional IVUS image wherein the radiological image further includes a calculated path within a vessel of interest;

FIG. 6 depicts an illustrative radiological image along-side a cross-sectional IVUS image wherein the radiological image further includes a calculated path within a vessel of interest with a marker positioned at a different location than the view of FIG. 5;

FIG. 7 depicts an illustrative enhanced radiological image along-side a cross-sectional IVUS image wherein the radiological image further includes a calculated path within a vessel of interest and a reference mark providing a point of synchronization/calibration of a marker position;

FIG. 8 depicts an illustrative catheter distal end including a single cylindrical radiopaque marker band;

FIG. 9 a depicts a radiopaque marker band 900, suitable for use in an exemplary embodiment, that partially encircles the catheter shaft;

FIG. 9 b depicts an imaging catheter having two of the radiopaque marker bands of the type depicted in FIG. 9 a wherein the two bands are skewed by a quarter rotation along the axis of the catheter;

FIG. 9 c depicts the imaging catheter of 9 b from a view that looks directly on the full surface of the distal marker band 920;

FIG. 9 d depicts the imaging catheter of 9 c at a view wherein the catheter is axially rotated 90 degrees from the position depicted in FIG. 9 c;

FIG. 9 e depicts the imaging catheter at a different rotational position from FIG. 9 c and FIG. 9 d; and

FIG. 10 is an exemplary flowchart describing a method 1000 for altering the image quality of an IVUS image using the pull-back speed.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to he understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

In accordance with embodiments of the present invention, a method and system are described by way of example herein below including image data acquisition systems that generate views on one or more displays that simultaneously provides positional information and intravascular images associated with an imaging probe (e.g., an intravascular ultrasound (IVUS) transducer probe) mounted upon a flexible elongate member (e.g., a catheter, guidewire, etc.). For example, sub-optimal image quality may prevent and/or hinder the diagnoses and/or treatment options of the vessels. As such, the methods and co-registration systems described below optimizes the image quality based on the detected pull-back speed of the imaging probe, which is determined from angiogram image data. For higher pull-back speeds, the system determines that a high frame rate is necessary to keep up and reduces the image quality. For slower pull-back speeds, the system determines that a lower frame rate is permitted, allowing for prioritizing resolution, depth of penetration, and/or other aspects of high quality images instead of frame rate. If the imaging probe is stopped, the system may recognize the zero motion of the imaging probe and select an even slower frame rate in order to achieve the highest image quality that the system can produce. In other words, the co-registration system helps produce images with the highest possible image quality for the necessary pull-back speed of the imaging probe, which helps the doctor and/or system make better diagnoses and/or more accurate treatment options (e.g., stent sizes) for the patient.

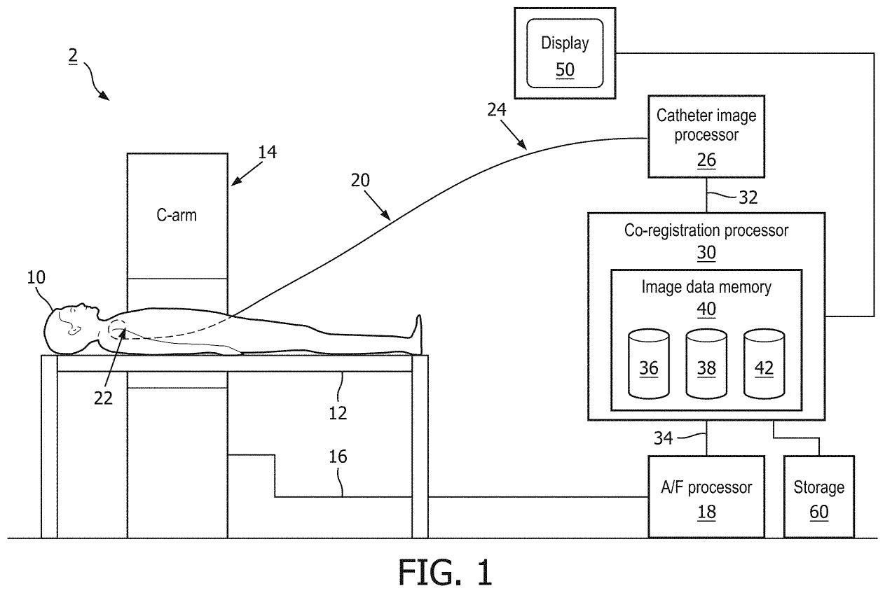

Turning initially to FIG. 1, an exemplary system 2 (co-registration system) is schematically depicted for carrying out the present invention in the form of co-registration of angiogram/fluoroscopy and intravascular ultrasound images. An example of a co-registration system is disclosed in U.S. Pat. No. 7,930,014 (filed Jan. 11, 2006, titled VASCULAR IMAGE CO-REGISTRATION), the entire disclosure of which is expressly incorporated by reference herein.

An angiogram device is used to capture the radiological image data. For example, a patient 10 is positioned upon an angiographic table 12. The angiogram device may include an angiographic table 12, an angiography/fluoroscopy unit c-arm 14, an angiograph/fluoroscopy processor 18, and/or a transmission cable 16. For example, the angiographic table 12 is arranged to provide sufficient space for the positioning of an angiography/fluoroscopy unit c-arm 14 in an operative position in relation to the patient 10 on the table 12. Radiological image data acquired by the angiography/fluoroscopy c-arm 14 passes to the angiography/fluoroscopy processor 18 via the transmission cable 16. The angiography/'fluoroscopy processor 18 converts the received radiological image data received via the cable 16 into angiographic/fluoroscopic image data. The angiographic/fluoroscopic (“radiological”) image data is initially stored within the processor 18.

Regarding portions of the system 2 associated with acquiring receiving ultrasound image data, an imaging catheter 20, and in particular an IVUS catheter, is inserted within the patient 10 so that its distal end, including a diagnostic probe 22 (in particular an IVUS imaging probe), is in the vicinity of a desired imaging location of a blood vessel. While not specifically identified in FIG. 1, a radiopaque material located near the probe 22 provides indicia of a current location of the probe 22 in a radiological image. For example, the diagnostic probe 22 generates ultrasound waves, receives ultrasound echoes representative of a region proximate the diagnostic probe 22, and converts the ultrasound echoes to corresponding electrical signals. The corresponding electrical signals are transmitted along the length of the imaging catheter 20 to a proximal connector 24. IVUS versions of the probe 22 come in a variety of configurations including single and multiple transducer element arrangements. In the case of multiple transducer element arrangements, an array of transducers is potentially arranged: linearly along a lengthwise axis of the imaging catheter 20, curvilinearly about the lengthwise axis of the catheter 20, circumferentially around the lengthwise axis, etc.

The proximal connector 24 of the catheter 20 is communicatively coupled to a catheter image processor 26. The catheter image processor 26 converts the signals received via the proximal connector 24 into, for example, cross-sectional images of vessel segments. Additionally, the catheter image processor 26 generates longitudinal cross-sectional images corresponding to slices of a blood vessel taken along the blood vessel's length. The IVIS image data rendered by the catheter image processor 26 is initially stored within the processor 26.

The type of diagnostic imaging data acquired by the diagnostic probe 22 and processed by the catheter image processor 26 may vary. In some examples, the diagnostic probe 22 is equipped with one or more sensors (e.g., Doppler and/or pressure) for providing hemodynamic information (e.g., blood flow velocity and pressure)—also referred to as functional flow measurements. The functional flow measurements are processed by the catheter image processor 26. It is thus noted that the term “image” is intended to be broadly interpreted to encompass a variety of ways of representing vascular information including blood pressure, blood flow velocity/volume, blood vessel cross-sectional composition, shear stress throughout the blood, shear stress at the blood/blood vessel wall interface, etc. In the case of acquiring hemodynamic data for particular portions of a blood vessel, effective diagnosis relies upon the ability to visualize a current location of the diagnostic probe 22 within a vasculature while simultaneously observing functional flow metrics indicative of cardiovascular disease. Co-registration of hemodynamic and radiological images facilitates precise treatment of diseased vessels. Alternatively, instead of catheter mounted sensors, the sensors may be mounted on a guidewire, for example a guidewire with a diameter of 0.018″ or less. Thus, in some instances, not only are a variety of probe types used, but also a variety of flexible elongate members to which such probes are mounted at a distal cud (e.g., catheter, guidewire, etc.).

A co-registration processor 30 receives INTIS image data from the catheter image processor 26 via line 32 and radiological image data from the radiological image processor 18 via line 34. Alternatively, the communications between the sensors and the processors are carried out via wireless media. The co-registration processor 30 renders a co-registration image including both radiological and IVUS image frames derived from the received image data. In sonic examples, indicia (e.g., a radiopaque marker artifact) are provided on the radiological images of a location corresponding to simultaneously displayed IVUS image data. The co-registration processor 30 initially buffers angiogram image data received via line 34 from the radiological image processor 18 in a first portion 36 of image data memory 40. Thereafter, during the course of a catheterization procedure IVUS and radiopaque marker image data received via lines 32 and 34 are stored within a second portion 38 and a third portion 42, respectively, of the image data memory 40. The individually rendered frames of stored image data are appropriately tagged (e.g., time stamp, sequence number, etc.) to correlate IVUS image frames and corresponding radiological (radiopaque marker) image data frames. In some examples, such as when the hemodynamic data is acquired rather than data, the hemodynamic data is stored within the second portion 38.

In addition, additional markers can be placed on the surface of the patient or within the vicinity of the patient within the field of view of the angiogram/fluoroscope imaging device. The locations of these markers are then used to position the radiopaque marker artifact upon the angiographic image in an accurate location.

The co-registration processor 30 renders a co-registration image from the data previously stored within the first portion 36, second portion 38 and third portion 42 of the image data memory 40. By way of example, a particular IVUS image frame/slice is selected from the second portion 38. The co-registration processor 30 identifies fluoroscopic image data within the third portion 42 corresponding to the selected IVUS image data from the second portion 38. Thereafter, the co-registration processor 30 superimposes the fluoroscopic image data from the third portion 42 upon the angiogram image frame retrieved from the first portion 36. Thereafter, the co-registered radiological and IVUS image frames are simultaneously displayed, along-side one another, upon a display device 50 (e.g., an LCD display device, an LED display device, and/or any other type of device able to display images). The co-registered image data frames driving the display device 50 are also stored upon a long-term storage device 60 for later review in a session separate from a procedure that acquired the radiological and IVUS image data stored in the image data memory 40.

While not shown in FIG. 1, a pull-back device is incorporated that draws the catheter 20 from the patient. Incorporation of such devices may facilitate calculating a current position of the probe 22 within a field of view at points in time when fluoroscopy is not active.

The co-registration processor 30 of FIG. 1 includes a non-transitory computer-readable medium (for example, memory), which includes instructions and/or logic that, when executed, cause the co-registration processor 30 to control the probe 20, the catheter image processor 26 and/or other components of the system 2. While three processors are shown in FIG. 1, in some examples, the functionalities of the catheter image processor 26, the co-registration processor 30, and/or the A/F processor 18 are performed by a single processor and/or distributed to one or more additional processors. For example, a single processor, such as the co-registration processor 30, may receive the ultrasound image data and/or the radiological image data and perform the functionalities of the three processors 26, 30, and 18 described above.

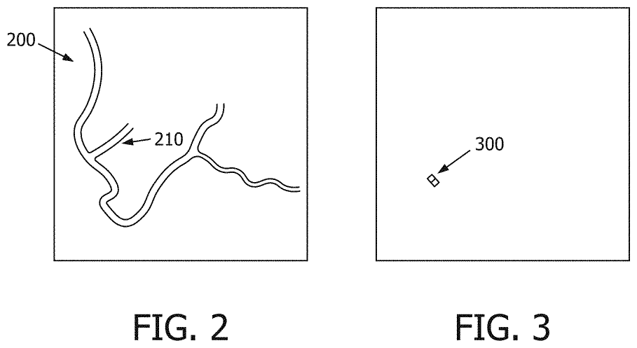

Turning to FIG. 2, the angiography/fluoroscopy processor 18 captures an angiographic “roadmap” image 200 in a desired projection (patient/vessel orientation) and magnification. For instance, in some examples, the image 200 is initially captured by an angiography procedure performed prior to tracking the IVUS catheter to the region of interest within a patient's vasculature. Performing the angiography procedure without the catheter 20 in the vessel provides maximal contrast flow, better vessel filling and therefore a better overall angiogram image. Thus, side branches such as side branch 210 and other vasculature landmarks can be displayed and seen clearly on the radiological image portion of a co-registered image displayed upon the graphical display device 50.

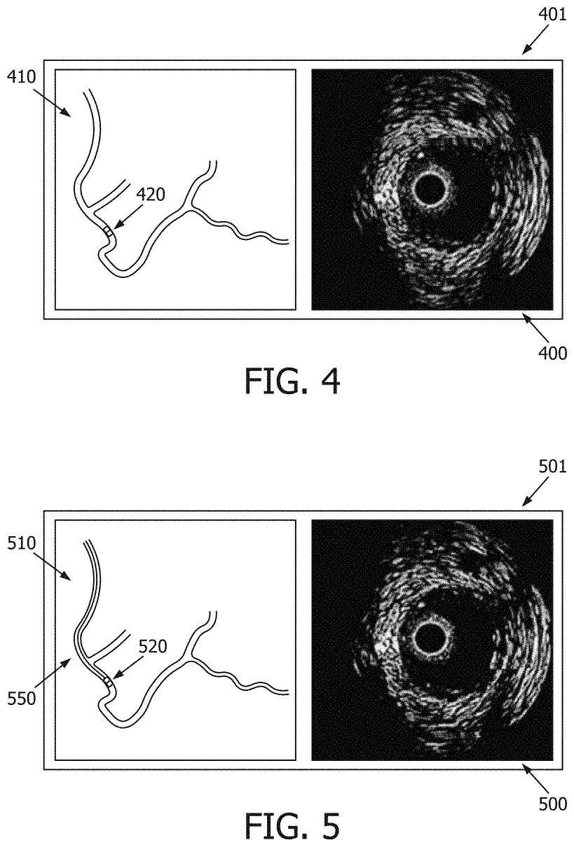

Turning to FIG. 3, the catheter 20 is tracked to its starting position (e.g., a region of interest or a position where an IVUS pull-back procedure begins). Typically, the catheter 20 is tracked over a previously advanced guidewire (not shown). Thereafter, a radiological image is obtained. In the image, the catheter radiopaque marker 300 is visualized, but the vessel lumen is not, due to the absence of contrast flow. However, a set of locating markers present in both the angiogram and fluoroscopy images enable proper positioning (superimposing) of the marker image within the previously obtained angiogram image. Other ways of properly positioning the radiopaque marker image within the field of view of the angiogram image are also considered and included herein. Furthermore, the marker artifact can be automatically adjusted (both size and position) on the superimposed image frames to correspond to the approximate position of the transducers. The result of overlaying/superimposing the radiopaque marker artifact upon the angiogram image is depicted, by way of example in an exemplary co-registration image depicted in FIG. 4.

Turning to FIG. 4 the exemplary co-registration display 401 (including the correlated radiological and IVUS images) depicts a selected cross-sectional IVUS image 400 of a vessel. A radiological image 410 is simultaneously displayed along-side the IVUS image 400 on the display 50. The radiological image 410 includes a marker artifact 420, generated from radiological image data rendered by a fluoroscope image frame, superimposed on an angiogram background rendered from the first portion 36 of the memory 40. The fluoroscope image frame corresponds to the current location of the diagnostic probe 22 within a vessel under observation. Precise matching of the field of view represented in both the angiogram and fluoroscope images (i.e., precise projection and magnification of the two images) allows identification of the current position of the IVUS probe corresponding to the displayed IVUS image 400 in the right pane of the co-registered images displayed in FIG. 4.

Alternatively, the composite radiological image 410 is obtained in one step. In such case, the original roadmap angiogram image is obtained with the catheter already in its starting position. However, once obtained, the angiogram image is reused as the IVUS probe is withdrawn from the vessel.

The system 2 also takes heart motion into account when generating/acquiring the radiological and IVUS image data. By way of example, by acquiring the image data for both the angiogram (background) and the radiopaque marker only during the peak R-wave of the EKG, heart motion is much less a factor and good overlay correlation exists between the angiogram and fluoroscope fields of view. The peak R-wave is selected because it represents end-diastole, during which the heart has the least amount of motion, and thus, a more consistent condition from which to obtain the radiological image data. The peak R-wave is also an easy point in the EKG for the system 2 to detect.

With continued reference to FIG. 4, in an exemplary embodiment when the IVUS catheter 20 begins to image, the cross-sectional image 400 from the IVUS catheter is displayed in tandem with the enhanced radiological image 410 including both the angiogram background and the superimposed marker artifact 420. The enhanced radiological image 410 and the cross-sectional IVUS image 400 are displayed close to (e.g., alongside) each other on the display 50, so that the operator can concentrate on the information in the cross-sectional image 400 while virtually simultaneously observing the status of the enhanced radiological image 410.

The simultaneous display of both the composite/enhanced radiological image and the cross-sectional image allows instant awareness of both disease state of a vessel segment and the location of the vessel segment within a patient. Such comprehensive information is not readily discernable in a three-dimensional flythrough image or a stacked longitudinal image. Neither flythrough nor stacked images alone allows for the simultaneous appreciation of 1) all of the information in a cross-section, 2) a feel for the shape of the vessel and 3) the location of the cross-section along the length of the vessel. The above-described “co-registration” of enhanced angiographic (including the marker artifact) and intravascular cross-sectional images/information delivers all three of these items in a presentation that is straight forward to an operator. The co-registration display is presented, by way of example, either on an IVUS console display, or the co-registration display is presented on one or more angiographic monitors, either in the room where the procedure is occurring or in a remote location. For example, one monitor over the table in the procedure room allows the attending physician to view the procedure, while at the same time a second consulting physician who has not scrubbed for the case is also able to view the case via a second monitor containing the co-registration display from a separate control room. Control room viewing is also possible without having to wear leaded covering.

Regarding the persistence of the background angiogram (“roadmap”) image portion of the enhanced radiological image 410, a single angiogram image is, by way of example, obtained/generated and stored in the first portion 36 of the memory 40 for a given procedure/patient position. If the field of view changes or the patient's position changes, then an updated background angiogram image is generated and stored in the first portion 36. Alternatively, the background angiogram image is live or continuously updated, for example, at each additional step in which angiography is performed. The projection of the angiogram roadmap/background image portion of the enhanced radiological image 410 is preferably in an orientation and magnification that best displays the entire vessel to be viewed, taking into account the foreshortening that is present in a tortuous/winding vessel. Alternatively, two roadmap images (or even two enhanced radiological images 410) can be used/displayed in place of the one image 410. Such multiple views are provided in the context of biplane angiography.

Establishing a position for the marker artifact within the field of the enhanced radiological image, based at least in part upon a radiopaque marker on the imaging catheter 20 is achievable in a variety of ways. Examples, described further herein below include: user-specified points (by clicking at a position near the marker to establish a point); image pattern recognition (automatic identification of a marker's unique signature within a field of view); and combinations of manual and automated calculations of a path.

Enhancing the background/roadmap angiogram image to render the image 410 is achieved in a number of different ways. As mentioned above, in an illustrative embodiment, the marker artifact 420 (derived from a fluoroscope image of a radiopaque marker near the probe 22 mounted on the distal end of the catheter 20) is superimposed upon/overlays the angiogram/roadmap background of the enhanced radiological image 410. Because the live/marker artifact portion of the image 410 requires that fluoroscopy be performed the entire time of catheter movement (e.g. pull-back), in an alternative embodiment, the marker artifact is displayed on the image 410 only during those periods when the fluoroscope is active. When the fluoroscope is inactive, only the background angiogram is presented on the enhanced image 410 of the display 50.

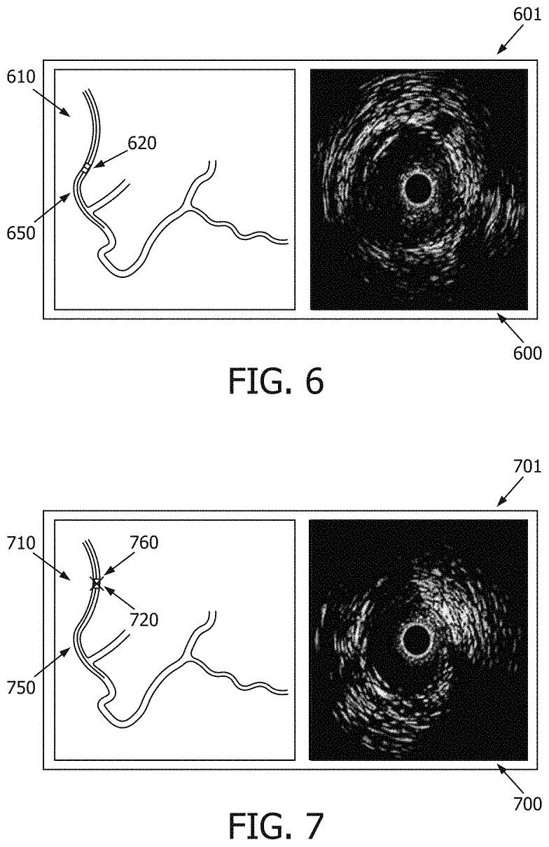

Turning to FIGS. 5 and 6, in embodiments of the invention, when the fluoroscope is inactive, the co-registration processor 30 calculates an approximate location of the radiopaque marker based upon its last registered position and other indicators of catheter movement (e.g., pull-back distance sensors/meters). The approximate location is utilized in place of the radiopaque marker image to render a marker artifact 520 on an enhanced radiological image 510 displayed along-side a corresponding IVUS cross-sectional image 500 within a display 501. By way of a particular illustrative example, during periods in which a fluoroscope is inactive, the marker artifact 520's position is calculated by software/hardware within the co-registration processor 30 from sensor data indicative of a current/changed location of the radiopaque marker within the current image field provided by the current background angiogram image. In an embodiment of the invention, a visual characteristic (e.g., color, symbol, intensity, etc.) of the marker artifact 520 is used to distinguish when the fluoroscope is active/inactive and thus indicate whether the marker artifact position is actual/calculated. Furthermore, in more advanced systems, both the displacement and angular orientation of the marker (and thus the diagnostic probe 22) are determined to render accurate approximations of the current position of the diagnostic probe 22 within a vessel as it acquires data for generating the image 500.

With continued reference to FIGS. 5 and 6, a calculated path 550/650 is determined by the co-registration processor 30 within displays 501/601. A marker artifact 520/620 is placed on top of the calculated path 550/650. The marker artifact 520/620 is superimposed on the angiogram image at a location calculated from non-visual position data (e.g., pull-back distance, spatial position sensors, angular orientation sensors, etc.), For example, if the initial location of a radiopaque marker within the enhanced radiological image 510/610 is known and the catheter is pulled by an automatic pull-back system at a specific rate for a known amount of time, the cursor can be placed by the system at a distance from the initial location along the calculated path 550/650 that represents the product of the pull-back rate and the time period. Furthermore, each subsequent time that a fluoroscope is activated and an image of the radiopaque marker is acquired and presented to the co-registration processor 30, an error between the actual radiopaque marker location and a current calculated marker artifact 520/620 location is eliminated by replacing the calculated position by a position calculated by the radiopaque marker image. The error between the corrected position and the calculated location of the marker artifact 520/620 is determined. In an exemplary embodiment, the error/total travel distance ratio is used as a scaling factor to recalculate and adjust all previously calculated/rendered/presented marker artifact overlay positions on the rendered/stored copies of the enhanced radiological image 510/610 for the entire preceding period in which the fluoroscope has been inactive.

Similarly, a recalculation can also update a shape of the calculated path 550/560 curve. As seen in FIGS. 5 and 6, the calculated path 550/650 is shown as a curve that matches the tortuosity of a vessel through which the probe 22 passes—represented by a center line through the displayed vessel. Alternatively, the catheter paths within vessels take a straighter and shorter path than the centerline of a blood vessel when pulled through such vessel. If, however, the catheter is being translated by pushing, instead of pulling, the calculated path 550/650 more closely matches the curvature of the vessel, or even exaggerates the tortuosity of the vessel by taking a longer path. A multiplication coefficient (e.g., 1.05 for pushing, 0.95 for pulling) can be introduced when calculating a path based upon this general observation of the path taken by a probe as it is pushed/pulled through a vessel. The path can alternatively be calculated from two different angiographic images taken at different projections (planes). This allows a three-dimensional angiographic image, from which a time centerline can be calculated.

In some examples, represented by the co-registered IVUS image 700 and enhanced radiological image 710 in a display 701 presented in FIG. 7, the operator creates a reference mark 760 at one or more points on a calculated path 750. The reference mark 760 serves a variety of potential uses. By way of example, the reference mark 760 potentially serves as a benchmark (location synchronization point) for updating position of a marker artifact 720 within the enhanced radiological image 710. In the embodiment represented by FIG. 7, the co-registration processor 30 waits for manual input of the reference mark 760 location information prior to proceeding with calculations. The user creates the reference mark 760 which coincides with a marker artifact 720 rendered from image data provided by a fluoroscope of a field of view containing a radiopaque marker. The reference mark 760, which potentially persists beyond its initial entry period, is distinguished from the marker artifact 720 which follows the current/estimated position of the probe 22. Furthermore, in an exemplary embodiment the reference mark is used to highlight/mark actual positions of the probe 22 (rendered by a fluoroscope image of a radiopaque marker) as opposed to estimated points on a calculated point (e.g. points on a path e.g., 550/560) from merely calculated position estimates upon the paths 550/560. In yet other embodiments, the reference mark 760 is used to highlight a particular point of interest during a diagnostic/treatment procedure. A bookmark is placed within a series of cross-sectional images associated with the IVUS image 700 portion of the display 701. The bookmark allows quick access to a particular archived image frame corresponding to the reference mark 760 in the display 701.

In some examples, a user interface associated with the displayed images provided in FIGS. 4-7 includes a “slider” control that allows an operator to track through a series of stored frames representing sequentially acquired data along a traversed path within a vessel. The slider control can he a set of arrows on a keyboard, a bar/cursor displayed upon an enhanced radiological image that can he manipulated by an operator, during playback, using a mouse or other user interface device to traverse a vessel segment, etc. For example, a display similar to FIG. 7 is rendered by the co-registration processor 30 during playback of a previous data acquisition session. A cursor similar to the reference mark 760 is displayed during playback on the enhanced radiological image 710. A user selects and drags the cursor along a path similar to the calculated path 750. As the user drags and drops the cursor along the path, the co-registration processor 30 acquires and presents corresponding co-registered images. The user sequentially proceeds through the stored images using, by way of example, arrow keys, mouse buttons, etc.

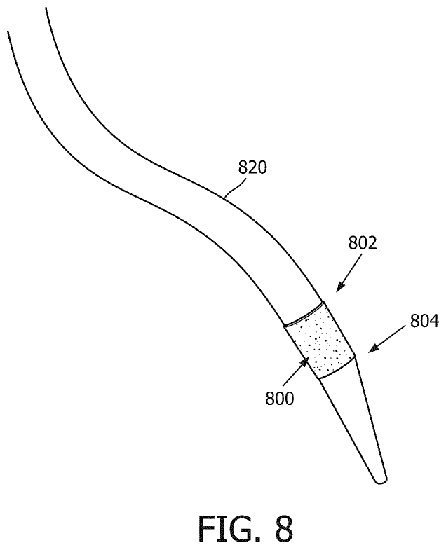

It is noted that various catheter marking schemes are contemplated that improve/optimize the co-registration processor 30's calculations of a position of the marker artifact (representing a position within a vessel corresponding to a currently displayed IVUS cross-section image) when the fluoroscope is inactive. Turning to FIG. 8, a single radiopaque marker band 800 is attached to the catheter 820 near an IVUS probe. The radiopaque band 800 includes a proximal edge 802 and a distal edge 804. The band 800 is cylindrical, with the diameter at the proximal edge 802 equal to the diameter at the distal edge 804. in addition, the band 800 has a known length.

Upon connection of the proximal connector 24 of the catheter 20 into an outlet on the catheter image processor 26 (or an interposed patient interface module which is communicatively connected to the processor 26), the processor 26 receives identification information from the catheter 20 via EPROM, RFID, optical reader or any other appropriate method for identifying the catheter 20. In an illustrative embodiment, the catheter length and diameter dimensions (or dimension ratio) are included in the received identification information. In addition, image field information such as magnification and/or projection angle) from the radiological image processor 18 is provided to the co-registration processor 30. By identifying four points at the corners of an approximate four-sided polygon of the marker band image, the co-registration processor 30 automatically calculates foreshortening of a vessel in an enhanced radiological image view and the true length of a segment of a calculated path.

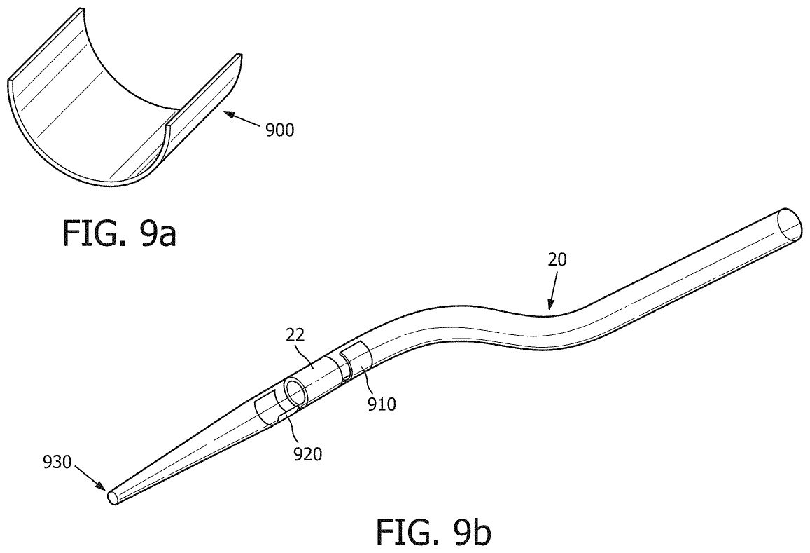

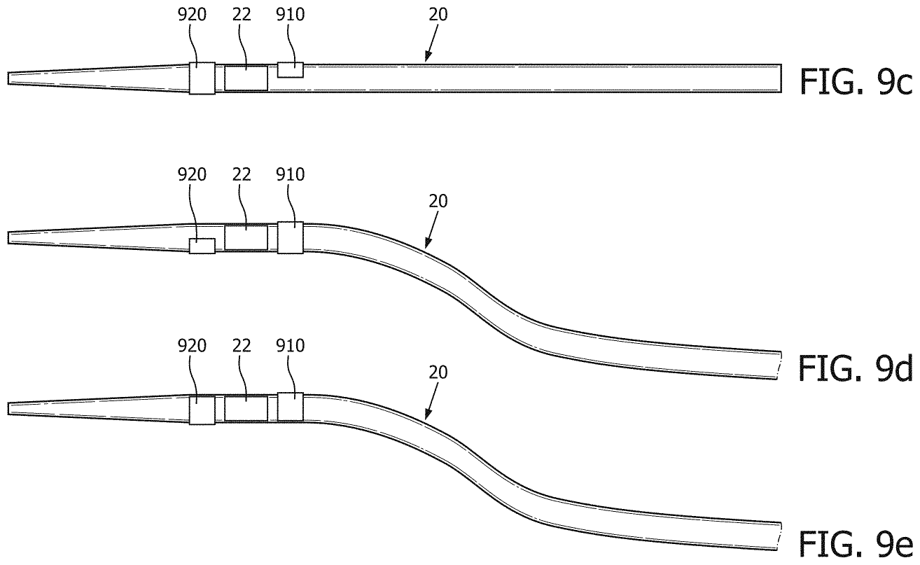

Turning briefly to FIGS. 9 a-e, a catheter 920 carries two marker bands having a known linear separation distance that facilitates making the calculations described herein above with reference to FIG. 8. FIG. 9 a shows a radiopaque marker band 900, suitable for use in an exemplary embodiment, that partially encircles the catheter shaft; In the exemplary embodiment, the marker band 900 extends about 180° (one half) of the perimeter of the catheter shaft. The band is potentially made, for example, of 100% Platinum, or 90% Platinum/10% Iridium, Tantalum, Gold or any other radiopaque materials or combinations/amalgams thereof.

FIG. 9 b shows an imaging catheter 20 having two of the radiopaque marker hands 910 and 920 of the type depicted in FIG. 9 a. The proximal band 910 is skewed 90° (a quarter of the circumference of the catheter 20) in relation to the distal band 920. In this embodiment, the bands 910/920 are shown equally spaced on opposite sides of the diagnostic probe 22. This catheter 20 also has a guidewire lumen 930 for passing a guidewire, for example a 0.014″ guidewire. The guidewire exits out the distal guidewire port. The proximal end of the guidewire can exit a proximal port either within the blood vessel (short lumen rapid exchange catheter), within a guiding catheter (long lumen rapid exchange catheter) or outside of the patient (over-the-wire catheter).

FIG. 9 c shows the imaging catheter 20 from a view that looks directly on the full surface of the distal marker band 920. Exactly one half of the proximal marker band 910, skewed by 90 degrees, is seen. An angiography image of the two marker bands, when viewed as shown in FIG. 9 c reveals band 920 having a thickness that is twice the thickness of the image of the band 910. Furthermore, an image length “L” of the marker bands 910/920 depends on angular position of the portion of the catheter 20 in the image containing the bands 910/920. In a perfect side view, the length L is equal to the actual length of the marker band. Offset O is equal to the difference between the thickness of band 920 and the thickness of band 910.

In FIG. 9 d an image is taken at a view wherein the catheter 20 is axially rotated 90 degrees from the position depicted in FIG. 9 c. The thickness of band 920 is half the thickness of band 910. Also, the position of the relative positions of the bands 910/920 in relation to the axis of the catheter 20 is used to determine the actual angular orientation of the catheter 20 since the offset alone is not enough to establish a current rotational position of the catheter 20.

FIG. 9 e is an image of the catheter 20 and bands 910/910 at a different rotational position from FIG. 9 c and FIG. 9 d. The orientation of the catheter can be determined by comparing the relative thicknesses (e.g., the offset, a ratio) of the thickness of images of the bands 910 and 920.

The descriptions hereinabove associated with illustrative embodiments using an IVUS catheter are applicable to a variety of alternative types of imaging catheters. Similarly, an enhanced radiological image can be combined with a longitudinal stack instead of a cross sectional slice—in fact, the enhanced radiological, transverse cross-sectional, and longitudinal cross-sectional images can be displayed together. In yet other embodiments, the enhanced radiological image is presented along-side an IVUS image including both grayscale and color image artifacts that characterizing tissue and deposits within a vessel. Additionally, the longitudinal IVUS grayscale image and/or the color (Virtual Histology) image are overlaid on the 2-D angiographic image or derived 3-D image.

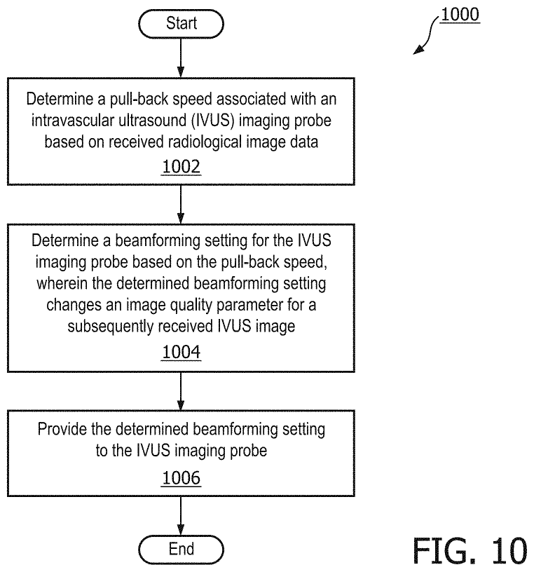

FIG. 10 shows a method 1000 for altering the image quality of an IVUS image using the pull-back speed and the angiogram image data. The co-registration processor 30 will he described below to perform the functionalities and/or steps of method 1000. However, in other examples, another processor, such as the catheter image processor 26, the A/F processor 18, a centralized processor, and/or one or more additional processors are used to perform one or more of the steps described below.

In operation, at step 1002, the co-registration processor 30 determines a pull-back speed of the IVUS imaging probe 22 and/or the catheter 20 based on received radiological image data. For example, as described above, the co-registration processor 30 receives an angiographic “roadmap” image data, such as data representing the image 200, indicating a region of interest (e.g., the vessel to be mapped and imaged by the system 2). Then, a user (e.g., doctor) may direct the catheter 20 and/or the imaging probe 22 to the region of interest within the patient 10 and in particular to a starting position.

The co-registration processor 30 receives a first radiological image data indicating a first position (e.g., starting position) of the catheter 20. For example, the catheter 20 includes one or more radiopaque markers, such as the radiopaque hand 800 shown in FIG. 8 and/or the radiopaque marker bands 910 and 920 shown in FIG. 9. The co-registration processor 30 causes the C-arm 14 to capture radiological image data indicating the location of the one or more radiopaque markers on the catheter 20. The co-registration processor 30 receives this first radiological image data and determines the starting position of the catheter 20.

Subsequently, the co-registration processor 30 receives a second radiological image data indicating a second position of the catheter 20. For example, the user may pull-back the catheter 20 and/or the imaging probe 22 and begin capturing IVUS images of the vessel such as image 400. After a pre-determined and/or pre-defined time period, the co-registration processor 30 causes the C-arm 14 to capture subsequent (e.g., second) radiological image data indicating the location of the radiopaque markers on the catheter 20. Additionally, or alternatively, the co-registration processor 30 causes the imaging probe 22 to capture ultrasound image data that is converted to an IVUS image (e.g., image 400 shown in FIG. 4). In some examples, the time period is stored in memory, such as memory 60, and the co-registration processor 30 uses the stored time period to capture the radiological image data and/or the ultrasound image data. Additionally, or alternatively, the co registration processor 30 receives user input (e.g., from the user) from a user input device (e.g., a graphical user interface, such as in examples where the display 50 is a touch screen, and/or an analog/digital user input) indicating the time period. In other words, the user may set the time period for the co-registration processor 30 to capture subsequent radiological image data and/or IVUS image data.

The co-registration processor 30 determines the pull-back speed based on the radiological image data (e.g., the first and second radiological image data). For example, the time period between images is known. The co-registration processor 30 determines a distance/length between the location of the radiopaque markers from the first and second radiological image data. Then, the co-registration processor 30 determines the pull-back speed based on the distance and time period.

In some examples, as explained above, the pull-back speed from the catheter 20 is known (e.g., the user may set the pull-back speed and/or it may he pre-determined such as by using an automated pull-back device) and the co-registration processor 30 uses the known pull-hack speed. For example, the system 2 may pull-back the catheter at a certain pull-back speed. The co-registration processor 30 might not calculate it using the radiological image data and may use the known pull-back speed.

At step 1004, the co-registration processor 30 determines a beamforming setting for the IVUS imaging probe 22 based on the pull-back speed. The beamforming setting indicates a number (e.g., frequency), angles, and/or the sequence of ultrasound pulses emitted by the imaging probe 22 and subsequently impacts the echoes of the pulses captured by the imaging probe 22. In other words, the beamforming setting is a pattern that the imaging probe 22 transmits and receives the ultrasound pulses/signals.

In at least one beamforming setting for a single IVUS image, the imaging probe 22 emits a number of ultrasound pulses. However, for increased pullback speeds, the imaging probe 22 might not have the opportunity to emit and receive that many pulses, which may impact image quality. In other words, the beamforming setting changes an image quality parameter of the IVUS image. For example, the beamforming setting affects one or more image quality parameters such as frame rate (e.g., more pulses emitted per frame means that each frame takes longer to create), resolution, depth of penetration, and artifacts. Reducing the number of frames per second allows each of those frames to have improved image quality. A high frame rate is needed to keep up with a fast pullback speed, but results in sub-optimal image quality for a slow pullback speed.

In some examples, if the user pulls the catheter 20 back quickly (e.g., a higher determined pull-back speed), the co-registration processor 30 determines that a high frame rate is beneficial in order to keep up and a lower beamforming setting (e.g., fewer ultrasound pulses are emitted). If the user pulls the catheter 20 back slowly (e.g., a slower determined pull-back speed), the co-registration processor 30 determines that a low frame rate is allowed and a higher beamforming setting (e.g., more ultrasound pulses are emitted). The higher beamforming setting allows the catheter to prioritize the image quality, such as the resolution, depth of penetration, or some other aspect of a high-quality image instead of frame rate. If the catheter 20 is stopped (e.g., a zero or substantially zero determined pull-back speed), the co-registration processor 30 selects an even slower frame rate and an optimal beamforming setting. (e.g., even more ultrasound pulses are emitted) in order to achieve the absolute highest image quality that the imaging probe 22 is able to produce. In other examples, the co-registration processor 30 uses additional, fewer, and/or different beamforming settings to affect one or more image quality parameters of the IVUS image.

At step 1006, the co-registration processor 30 provides the determined beamforming setting at step 1004 to the IV US imaging probe 22. The imaging probe 22 uses the determined beamforming setting to emit the ultrasound pulses and/or capture the echoes from the pulses. Then, as explained above, the catheter image processor 26 generates an IVUS image based on the pulses and provides it to the co-registration processor 30. The co-registration processor 30 provides for display on the display 50 the IVUS image that was captured using the determined beamforming setting. Additionally, or alternatively, similar to FIGS. 4-7, the co-registration processor 30 also displays the radiological image with the IVUS image.

The co-registration processor 30 may constantly update the beamforming setting based on the pull-back speed of the catheter 20 and/or the imaging probe 22. For example, after each pre-determined, pre-programmed, and/or user-defined time period, the co-registration processor 30 receives another radiological image data and compares it to the previous radiological image data to determine the pull-back speed. The co-registration processor 30 determines a new beamforming setting and then provides it to the imaging probe 22. Then, the co-registration processor 30 provides for display the new IVUS image with the new beamforming setting, and the method 1000 repeats.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain fights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A medical imaging system including a co-registration system comprising:
one or more processors;
an intravascular ultrasound (IVUS) device comprising an IVUS imaging probe, the IVUS device in communication with the one or more processors;
an angiogram device in communication with the one or more processors; and
memory storing instructions that when executed by the one or more processors, cause the one or more processors to:
determine a pull-back speed of the IVUS imaging probe based on received radiological image data from the angiogram device;
determine a beamforming setting for the IVUS imaging probe based on the pull-back speed, wherein the determined beamforming setting changes an image quality parameter for a subsequently received IVUS image; and
provide the determined beamforming setting to the IVUS imaging probe.

2. The medical imaging system including the co-registration system of claim 1, wherein the IVUS imaging probe comprises one or more radiopaque markers, wherein the received radiological image data comprises first radiological image data indicating a first location of the one or more radiopaque markers and second radiological image data indicating a second location of the one or more radiopaque markers, and wherein the determining the pull-back speed is based on the first location and the second location.

3. The medical imaging system including the co-registration system of claim 2, wherein the determining the pull-back speed of the IVUS imaging probe comprises:
determining the pull-back speed based on determining a distance between the first location and the second location.

4. The medical imaging system including the co-registration system of claim 1, wherein the memory storing instructions that when executed by the one or more processors, further cause the one or more processors to:
receive a user input indicating the pull-back speed, and
wherein the determining the pull-back speed is based on the user input.

5. The medical imaging system including the co-registration system of claim 1, wherein the beamforming setting indicates a number and sequence of ultrasound pulses emitted by the IVUS imaging probe, and
wherein the image quality parameter for the subsequently received IVUS image is based the number and sequence of the ultrasound pulses emitted by the IVUS imaging probe.

6. The medical imaging system including the co-registration system of claim 1, wherein the image quality parameter comprises at least one of frame rate, resolution, and depth of penetration of a vessel.

7. The medical imaging system including the co-registration system of claim 1, wherein the determining the beamforming setting comprises determining a first beamforming setting based on a first pullback speed, wherein the first beamforming setting indicates a first number of ultrasound pulses emitted by the IVUS imaging probe.

8. The medical imaging system including the co-registration system of claim 7, wherein the determining the beamforming setting comprises determining a second beamforming setting based on a second pullback speed, wherein the second pullback speed is slower than the first pullback speed, wherein the second beamforming setting indicates a second number of ultrasound pulses emitted by the IVUS imaging probe, and wherein the second number of ultrasound pulses is greater than the first number of ultrasound pulses.

9. The medical imaging system including the co-registration system of claim 8, wherein the determining the beamforming setting comprises determining a third beamforming setting based on a pullback speed indicating substantially zero. wherein the third beamforming setting indicates an optimal number of ultrasound pulses emitted by the IV US imaging probe, and wherein the optimal number of ultrasound pulses is greater than the first number of ultrasound pulses and the second number of ultrasound pulses.

10. The medical imaging system including the co-registration system of claim 1, further comprising:
a display device; and
wherein the memory storing instructions that when executed by the one or more processors, further cause the one or snore processors to:
receive subsequently received IVUS image data based on the determined beamforming setting;
generate the subsequently received IVUS image from the subsequently received IVUS image;
generate a subsequently received radiological image corresponding to the subsequently received IVUS image; and
provide for display on the display device, the subsequently received IVUS image and the subsequently received radiological image.

11. A non-transitory computer readable medium storing instructions for execution by a processor incorporated into a medical imaging system including a co-registration system, wherein execution of the instructions by the processor cause the processor to:
determine a pull-back speed of an intravascular ultrasound (IVUS) imaging probe based on received radiological image data from an angiogram device;
determine a beamforming setting for the IVUS imaging probe based on the pull-back speed, wherein the determined beamforming setting changes an image quality parameter for a subsequently received IVUS image; and provide the determined beamforming setting to the IVUS imaging probe.

12. The non-transitory computer readable medium of claim 11, wherein the IVUS imaging probe comprises one or more radiopaque markers, wherein the received radiological image data comprises first radiological image data indicating a first location of the one or more radiopaque markers and second radiological image data indicating a second location of the one or more radiopaque markers, and wherein the determining the pull-back speed is based on the first location and the second location.

13. The non-transitory computer readable medium of claim 12, wherein when determining the pull-hack speed of the IVUS imaging probe, the instructions cause the processor to:
determine the pull-back speed based on determining a distance between the first location and the second location.

14. The non-transitory computer readable medium of claim 11, the instructions further causing the processor to:
receive a user input indicating the pull-back speed, and
wherein the pull-back speed is based on the user input.

15. The non-transitory computer readable medium of claim 11, wherein the beamforming setting indicates a number and sequence of ultrasound pulses emitted by the IVUS imaging probe, and
wherein the image quality parameter for the subsequently received IVUS image is based the number and sequence of the ultrasound pulses emitted by the IVUS imaging probe.

16. The non-transitory computer readable medium of claim 11, wherein the image quality parameter comprises at least one of frame rate, resolution, and depth of penetration of a vessel.

17. The non-transitory computer readable medium of claim 11, wherein when determining the beamforming setting, the instructions cause the processor to determine a first beamforming setting based on a first pullback speed, wherein the first beamforming setting indicates a first number of ultrasound pulses emitted by the IVUS imaging probe.

18. The non-transitory computer readable medium of claim 17, wherein when determining the beamforming setting, the instructions cause the processor to determine a second beamforming setting based on a second pullback speed, wherein the second pullback speed is slower than the first pullback speed, wherein the second beamforming setting indicates a second number of ultrasound pulses emitted by the IVUS imaging probe, and wherein the second number of ultrasound pulses is greater than the first number of ultrasound pulses.

19. The non-transitory computer readable medium of claim 18, wherein when determining the beamforming setting, the instructions cause the processor to determine a third beamforming setting based on a pullback speed indicating substantially zero, wherein the third beamforming setting indicates an optimal number of ultrasound pulses emitted by the IVUS imaging probe, and wherein the optimal number of ultrasound pulses is greater than the first number of ultrasound pulses and the second number of ultrasound pulses.

20. The non-transitory computer readable medium of claim 11, the instructions further causing the processor to:
receive subsequently received IVUS image data based on the determined beamforming setting;
generate the subsequently received IVUS image from the subsequently received IVUS image;
generate a subsequently received radiological image corresponding to the subsequently received IVUS image; and
provide for display on a display device, the subsequently received IVUS image and the subsequently received radiological image.
